(19) United States
(12) Patent Application Publication
(10) Pub. No.: US 2010/0034698 A1
(43) Pub. Date: Feb. 11, 2010

(54) METHOD FOR HEAT TREATMENT AND PROCESSING OF BIOLOGICAL MATERIALS

(76) Inventor: Steinar Gjersdal, Volda (NO)

Correspondence Address: ROTHWELL, FIGG, ERNST & MANBECK, P.C., 1425 K STREET, N.W., SUITE 800, WASHINGTON, DC, 20005, US

(21) Appl. No.: 12/444,921

(22) PCT Filed: Oct. 9, 2007
(86) PCT No.: PCT/NO07/00354

§ 371 Date: May 14, 2009

(30) Foreign Application Priority Data
Oct. 9, 2006 (NO) 20064604

(51) Int. Cl. A61L 2/00

(52) U.S. Cl. 422/38

(57) ABSTRACT

A method is provided for heat-treatment and processing of biological materials with a fluid from a fluid source in a closed packaging/container, wherein the fluid is introduced into the packaging/container for direct treatment contact with the biological materials and the fluid is transported or circulated out of the packaging, thereafter packaging/container is completely sealed. The method may be used for heat-treatment and processing of biological materials with a fluid in order to increase the shelf life or the sensoric qualities of the material.

The present invention relates to a method for heat treatment and processing of a biological material or materials with a fluid from a fluid source in a closed package.

### BACKGROUND OF THE INVENTION

The quality and shelf life of biological materials are reduced partly because of the microbial activity of, for example, bacteria, fingi and mould.

It is known to extend the shelf life of a biological material by adding a gas mixture of, e.g., carbon dioxide and nitrogen in closed packaging in order, inter alia, to inhibit growth of micro-organisms.

A method of treating oxygen-sensitive material with carbon dioxide in closed packaging is known from U.S. Pat. No. 6,023,915. The oxygen in the packaging is displaced by carbon dioxide before the packaging is sealed.

From U.S. Pat. No. 3,521,806 there is known a method for treating perishable products such as food or flowers with an inert gas in closed packaging, wherein air is first evacuated from the packaging. The packaging is then pressurised with inert gas and sealed for dispatch and storage.

The said methods do not provide any possibility of efficiently killing bacteria and other micro-organisms or of enhancing the sensory properties of the biological material.

It is known to heat treat food products in steam in a closed package, where the steam is produced internally in a package of, for example, aluminium (with some water in the bottom) by directly exposing the package to an external heat source (hot plate, gas burner, microwave oven) whereby an internal generation of steam occurs within the package, the steam being released from the packaging through a valve, and where this process takes place under normal atmospheric pressure. The treatment thus takes place solely with steam at atmospheric pressure. and it provides a heat treatment at about 100 degrees.

This method does not provide any possibility of controllably adjusting (e.g., increasing) pressure, temperature, saturation, quantity and/or circulation or of adding other gases or liquids than the inherent water vapour for a more efficient treatment.

The present invention solves the aforementioned limitations in an efficient manner by using an external fluid source in a tubing system that is brought into direct contact with the biological material or materials in the closed packaging and is then circulated or evacuated back out of the packaging.

The term “biological material” in connection with the present invention is to be understood as meaning raw products, semi-prepared products or ready-made dishes, diets etc. for human and animal nutrition.

It should be stressed that the invention may also be used advantageously in connection with other biological material such as various types of biological waste.

The use of an external fluid source allows the fluid to be any gas or liquid, approved for the particular use, that can be brought to a desired pressure, temperature, saturation, quantity, composition and circulation etc. before it is introduced into the packaging where it comes into direct contact with the biological material that is to be treated, after which the fluid is transported out of the packaging.

The packaging may, for example, be a bag, pot, tub, plastic bucket, plastic drum, industrial container, for example a pallet container of plastic or the like, of soft/flexible shape or rigid shape, and may be produced of all types of heat-resistant materials.

The packaging is designed to be closed after the foods have been placed therein, whereupon fluid is added to the packaging and treatment is carried out within the packaging, after which the packaging is sealed.

The method for treatment in a closed package according to the invention is thus characterised in that the fluid is introduced into the package for direct treatment contact with biological material or materials, and the fluid is transported out of the packaging, and once treatment has been completed, the packaging is sealed.

### DETAILED DESCRIPTION OF THE INVENTION

### The Method

The invention is intended to be a part of an industrial process wherein the biological material is first processed or prepared and then placed in packaging that is closed. The method according to the invention is then carried out, after which the packaging is completely sealed and then the packaging is typically stored and transported to the users.

The packaging may, for example, be a soft/flexible bag or pot or the like, or the packaging may be in rigid form such as, e.g., a tub, bucket, container/industrial container or the like, and where the packaging is adapted for different users such as an individual, a large household, catering services, an institution, industry etc. The packaging will be made from all types of heat-resistant materials and be of any material that is approved for use in the packaging of biological materials, which can be heated up. Such packaging materials may, for example, be plastic, aluminium, polystyrene foam or glass.

The biological material may be a foodstuff as, for instance, fish, meat, shellfish, vegetables, potatoes, pasta, rice, pulses, spices, herbs, fruit, berries etc., which may be fresh or processed, for example, by freezing, drying, marinating, boiling, steaming etc.

In many cases, the biological material will be a composite food product which, after processing by means of the method according to the invention, will be fully steam-cooked and ready for eating, but can, if desired, be heated in an oven, microwave oven, or pan, or fried in a frying pan, wok or the like in connection with a meal.

The users may, for example, be private individuals, eating and drinking establishments, institutions, industrial plants or the like that purchase the product.

The shelf life of the biological material will depend upon what it consists of and how the method is carried out.

If the biological material is a food product and a fresh food, it will be cold-stored, usually for a couple of weeks or more.

If the biological material is a food product that is not a fresh food and, for example, is treated with fluid prepared under high pressure and at a high temperature and a sterile gas, the storage time will be even longer.

During the performance of the method, the biological material will come into direct contact with the fluid. This may be a hot gas, hot liquid vapour and/or hot liquid.

The term “hot” should be understood here to mean a temperature that is above ambient temperature, preferably above 50° C., and most preferably around 100° C. or higher.

A hot gas may have a variable composition and temperature and may contain components that help to preserve or prolong the shelf life of the biological material, or it may add other desired properties.

For example, a gas mixture without oxygen may result in reduced oxidation of, inter alia, fats and thus less rancidity. A hot gas may, e.g., kill micro-organisms and dry out the surface of biological materials.

A hot liquid vapour may be of varying pressure, saturation and temperature and produced from all types of liquids, for example, a liquid may have taste additives added thereto with which the biological material can advantageously be treated. A hot liquid vapour can, e.g., kill micro-organisms and dry out the surface of the biological materials.

A hot liquid may be of varying chemical composition and temperature, and may contain components which help to preserve or prolong shelf life of the biological material, or it may add other desirable properties. A hot liquid may, for example, have taste additives added thereto with which the biological material may advantageously be treated and also remain stored in.

During the performance of the method, the fluid can be circulated into and out of the packaging through, for example, valves, tube connections, tubes, hollow needles or the like. Valves and tube connections may be of different designs and will be a part of the actual packaging structure. Tubes/hollow needles will be a part of the actual fluid system and form temporary inlets/outlets in the packaging for circulating fluid, where the performance of the method, for example, can take place in connection with a packing machine.

If the packaging is a soft/flexible bag of a plastic material, for example, film, the fluid can be introduced into the packaging, for example, through a tube/hollow needle that is inserted between the plastic films of which the packaging is composed. The fluid is then transported out of the packaging in a similar way.

If rigid packaging such as a bucket, drum, industrial container or the like is used, the fluid could be circulated into and out through, e.g., valves or tube connections belonging to the actual packaging structure.

In some embodiments, the biological material can advantageously be raised slightly from the bottom of the packaging by means of, e.g., a loose rack or ridges or the like with which the packaging structure is equipped.

The fluid is prepared from an external fluid source where the desired pressure, temperature, saturation degree and quantity can be obtained before the fluid is added to the biological material in the packaging.

The fluid source may be any source that can be connected to the system, such as a gas tank.

The fluid source is preferably a heating boiler/steam boiler for preparing hot gas, hot liquid and/or hot liquid vapour at different pressures and temperatures, saturation and quantities.

The pressure produced therein may be positive pressure, atmospheric pressure or negative pressure.

By increasing or lowering the pressure in the fluid source, it is possible to obtain a gas, liquid or vapour pressure that is optimal for the treatment of the individual biological material.

Some types of biological material will, for example, be given a better treatment and texture at a higher pressure. By increasing or lowering the pressure, it will also be possible to produce vapour of a liquid having respectively a raised or lowered temperature in relation to its temperature at normal atmospheric pressure. Thus, it is possible to treat some types of meat at a temperature higher than 100° C., which may give a better result than ordinary steam cooking.

It is of course advantageous to be able to vary the temperature of the fluid for adaptation to different biological materials.

By increasing the temperature of liquid vapour to more than 100° C. by increasing the pressure in the vapour source, it is possible to obtain a more efficient treatment of the biological material than with ordinary water vapour/liquid vapour at 100 C.

According to a preferred embodiment of the method according to the invention, the pressure is increased beyond atmospheric pressure for a given period to obtain a more efficient treatment of the biological material.

The treatment time will vary according to the type of biological material that is treated, and will increase with larger quantities and/or volumes and be reduced at higher vapour pressure (e.g., higher temperature and saturation) and higher vapour quantity and vapour circulation.

In general, the treatment time with fluids of a different nature (different composition, pressure, temperature, quantity, degree of saturation etc.) will of course vary depending on the different biological materials and the desired results.

Table 1 below shows how pressure and temperature are dependent upon each other for saturated water vapour.

TABLE 1
Pressure-temperature for saturated water
vapour based on 1 atmospheric pressure

| BAR | ° C. |
|---|---|
| 1 | 120 |
| 2 | 133 |
| 3 | 143 |
| 4 | 151 |
| 5 | 158 |
| 6 | 165 |
| 7 | 171 |
| 8 | 175 |
| 9 | 179 |
| 10 | 184 |
| 11 | 188 |
| 12 | 192 |
| 13 | 196 |
| 14 | 199 |
| 15 | 202 |
| 16 | 204 |
| 17 | 207 |
| 18 | 209 |
| 19 | 212 |
| 20 | 216 |
| 22 | 221 |
| 24 | 225 |

When the fluid has left the fluid source, it will be passed into the packaging. Depending on how this is done, the temperature and the pressure of the fluid can be altered slightly before the fluid enters the packaging. For example, the temperature of the fluid can be lowered slightly during the transport. If the fluid is prepared at positive pressure, the pressure can be lowered if desirable, for example, to avoid damage to some types of packaging. This can be done using known means and methods.

For example, the fluid can be passed via a hose into a large container such as a storage tank so that the pressure is lowered, and then transported further by a hose having a diameter that is slightly larger than the hose that passed the fluid into the container, so that the temperature can be maintained above the temperature usually obtained with the particular fluid at atmospheric pressure even if the pressure is lowered until it is atmospheric.

According to one particular preferred embodiment of the method, the packaging is thus subjected only to atmospheric pressure or approximately atmospheric pressure as fluid that is produced having positive or negative pressure is pressure-normalized before it is introduced into the packaging.

The packaging can also be protected directly from positive pressure if desired. The packaging can then be placed inside a pressure-resistant dome with internal dimensions that are equal to or less than the maximum dimensions of the packaging, so that the packaging cannot be expanded to more than its desired maximum size even when high pressure is used.

The packaging can also be protected directly from positive pressure or negative pressure by being placed inside a pressure-resistant chamber where it is put under pressure or vacuum in order to neutralise the positive pressure or negative pressure.

If the packaging is placed in a pressure-resistant dome or a chamber that is put under pressure or vacuum, pressure equalisation of the vapour before it is introduced into the packaging is avoided.

When the fluid is introduced into the packaging, it will displace and replace the air in the packaging and come into direct contact with the biological material and will thus treat the material, the fluid then being transported or circulated out of the packaging. A continuous stream and volume control of the fluid towards the biological material is thus obtained.

An alternative method is to use a soft/flexible packaging (e.g., a bag), where the fluid is alternately injected into and evacuated out of the packaging.

This method can be used if there is a need for the fluid to surround the biological material for a short time (optionally a few times) and not circulate towards it over time.

This method can also be carried out using fluid at positive or negative pressure, in that the packaging is placed in a pressure-resistant chamber.

This method can be used in the treatment of biological material which is only to have a short treatment in order, for example, to kill any surface bacteria that may have developed in connection with the packing process.

When the fluid source is ultimately disconnected from the packaging, the packaging is sealed completely using known means. Sealing the packaging may, for example, be done using plastic film and heat (e.g., heat sealing) or mechanical means such as valves or the like depending upon the type of packaging material and packing method that are chosen in connection with the performance of the method.

If the sealing of the packaging takes place once the treatment is terminated, no air from outside will introduce bacteria and other micro-organisms or oxygen after the treatment. If the biological material has been sterilised during the treatment, it will thus remain sterile.

Before the packaging is sealed, it may optionally be treated further.

One form of additional treatment may be to cool the biological material by circulating a cold fluid within the packaging. By “cold” is meant a fluid having a lower temperature than the packaging and the biological material. This may be the same fluid or fluids as that or those the biological material was treated with, or another fluid. For example, cold water vapour or nitrogen can be used from a separate container.

Another form of additional treatment may be to circulate a fluid having desired properties which is to surround the biological material during its future storage. This may, for example, be an inert gas which by not containing oxygen prevents oxidation, or a liquid which, for example, is to protect the texture of the biological material. This is added from a separate container.

Another possible additional treatment is to remove gas and/or process fluid (e.g., condensation water) from the packaging. The gas and/or process liquid can be sucked or pressed out of the packaging by, e.g., applying vacuum or pressure. Removal of process liquid can preferably be effected through valves or tube connections with which the packaging structure is equipped.

If the packaging is sealed immediately after completed treatment with liquid vapour, a vacuum will arise in the packaging during external cooling. If the packaging is made having at least one part capable of collapsing, for example, a top part of soft plastic (for example, “slack” plastic film) or the packaging is, e.g., a bag, the packaging can then rest quite snugly directly on the biological material.

### Uses of the Method

As described above, a number of different biological materials can be treated using the method according to the invention. For example, ready-to-eat dishes and hot dishes can be prepared, such as steamed vegetable and potatoes, steamed chicken or fish fillet etc. with an enhanced texture and flavour.

The enhancement of sensory properties can be measured using scientifically recognised methods, such as by a taste panel. Examples of sensory properties include properties such as smell, taste and/or texture as well as appearance.

The use of the method according to the invention thus gives a desired texture, flavour and quality of the food products/biological materials by optimising the different parameters of the treatment (pressure, temperature, time, vapour quantity etc.).

The biological materials may be semi- or fully through-treated or merely surface-treated, depending on the temperature of the fluid and the duration of the treatment, according to the result that it is desired to obtain.

The use of the method according to the invention also results in an improved shelf life when seen in comparison with a similar product that has not been prepared using hot gas, hot vapour and/or hot liquid directly against the biological material in closed packaging.

As stated above, this is due to the fact that no air or micro-organisms get into the packaging after the treatment has been completed, as the packaging is sealed immediately after the treatment is terminated.

Methods for measuring sealed shelf life are known in the field, and include techniques such as measuring bacteria levels and measuring oxidation, and will vary according to the nature of the biological material.

1-12. (canceled)

13. A method for direct heat treatment, processing and sterilization of biological material(s), placed inside a heat-resistant packaging including inlet(s) and/or outlet(s), by means of a hot fluid from an external fluid source, said method comprising the steps of:
a) enclosing the biological material(s) in the packaging, thereby separating the biological material(s) from possible non sterile environment outside the packaging,
b) introducing fluid into the closed package;
c) treating the biological material(s) inside the package with said fluid; and
d) sealing the inlet(s) and/or outlet(s) in the package, thereby creating a sealed chamber with a sterile environment.

14. The method according to claim 13, wherein the fluid is introduced into the closed package through inlet(s).

15. The method according to claim 13, wherein the hot fluid is gas, liquid vapor and/or liquid.

16. The method according to claim 13, wherein the biological material(s) is/are foodstuff for human nutrition treated in order to enhance shelf life and/or sensory properties.

17. The method according to claim 13, wherein the biological material(s) is/are biological waste for animal nutrition treated in order to enhance shelf life and/or sensory properties.

18. The method according to claim 13, wherein the fluid is preprocessed by heating in the external fluid source producing a pressure that may differ from atmospheric pressure and that is varied so that the properties of the fluid, such as pressure, temperature, saturation, quantity etc. are optimized in relation to the specific biological material(s) being treated.

19. The method according to claim 13, wherein the fluid pressure is increased beyond atmospheric pressure in order to prepare fluid having a temperature above 100° C., or that the fluid pressure is lowered below atmospheric pressure to prepare fluid having a temperature below 100° C., whereby the fluid pressure can optionally be normalized after the fluid has been treated and before the fluid is introduced into the packaging.

20. The method according to claim 13, wherein the packaging is placed in a pressure resistant chamber whereupon a pressure or vacuum is applied inside the chamber to avoid possible damage of the packaging using fluid with positive pressure or negative pressure.

21. The method according to claim 13, wherein the biological material(s) is/are cooled after heat treatment by introducing a cold fluid into the packaging.

22. The method according to claim 13, wherein the biological material(s) is/are surrounded by liquid or gas to prevent oxidation and to protect the texture of the biological material(s) during future storage.

23. The method according to claim 14, wherein said inlet(s) comprise one or more of valve(s), tube(s), and needle(s).
